SEQUENCE LISTING (1 sequence)

SEQ ID NO: 1 - protein, 27 aa, Homo sapiens
CCCCCACGTTGTAAAACGACGGCCAGT

(19) United States
(12) Patent Application Publication
(10) Pub. No.: US 2008/0153085 A1
(43) Pub. Date: Jun. 26, 2008

(54) Optical Detection of Analytes by Use of Semiconductor Nanoparticles

(76) Inventors: Fernando Patolsky, Rehovot (IL); Ron Gill, Jerusalem (IL); Yossi Weizmann, Ramat Hagolan (IL); Itamar Willner, Mevasseret Zion (IL); Uri Banin, Mevasseret Zion (IL); Taleb Mokari, Jerusalem (IL)

Correspondence Address: NATH & ASSOCIATES, 112 South West Street, Alexandria, VA, 22314, US

(21) Appl. No.: 10/566,078

(22) PCT Filed: Jul. 28, 2004
(86) PCT No.: PCT/IL04/00692

§ 371 Date: Jan. 30, 2008

Related U.S. Application Data
(60) Provisional application No. 60/490,259, filed on Jul. 28, 2003.

(51) Int. Cl. C12Q 1/68; G01N 27/00; C12Q 1/30; G01N 21/66; B01J 19/00; G01N 33/00

(52) U.S. Cl. 435/6; 436/149; 436/94; 435/27; 422/68.1; 422/82.08; 977/774

(57) ABSTRACT

The present invention provides an optical method and device for the determination of an analyte in an assayed sample. The method and device of the invention are based on the use of semiconductor nanoparticles that carry a recognition agent. The detection is based on fluorescence resonance energy transfer (FRET) between the semiconductor nanoparticle donors, which are excited with electromagnetic radiation, and acceptors in the form of dye-labeled or semiconductor nanoparticle-labeled agents, that are immobilized to the recognition agent in the presence of the analyte and under assay conditions. The method and device of the present invention are especially useful in the determination of DNA or RNA analytes, DNA polymerase or telomerase analytes, cancer cells through telomerase activity and single-base mutations. In such cases the recognition agent is a single-stranded oligonucleotide.

### FIELD OF THE INVENTION

This invention relates to an analytical method and device for the determination of the presence and/or the concentration of an analyte in a liquid medium. More specifically, the present invention concerns a fast and sensitive optical method for the detection of cancer cells, DNA analyte or single base mutations.

### LIST OF REFERENCES

The following references are considered to be pertinent for the purpose of understanding the background of the present invention.

1. Bruchez, M.; Moronne, M.; Gin, P.; Weiss, S.; Alivisatos, A. P. Science 1998, 281, 2013-2016.

2. Mattoussi, H.; Mauro, J. M.; Goldman, E. R.; Anderson, G. P.; Sundar, V. C.; Mikulec, F. V.; Bawendi, M. G. J. Am. Chem. Soc. 2000, 122, 12142-12150.

3. Mao, C.; Sun, W.; Seeman, N. C. Nature 1999, 397, 144-146.

4. Yurke, B.; Turberfield, A. J.; Mills, Jr. A. P.; Simmel, F. C; Neumann, J. L. Nature 2000, 406, 605-608.

5. Tan, W.; Fang, X.; Li, J.; Liu, X. Chem. Eur. J. 2000, 6, 1107-1111.

6. Dubertret, M.; Calame, M.; Libchaber, A. J. Nat. Biotechnol. 2001, 19, 365-370.

7. Patolsky, F.; Lichtenstein, A.; Kotler, M.; Willner, I. Angew. Chem. Int. Ed. 2001, 40, 2261-2264.

8. Patolsky, F.; Weizmann, Y; Willner, I. J. Am. Chem. Soc. 2002, 124, 770-772.

9. Bruchez, M.; Moronne, M.; Gin, P.; Weiss, S.; Alivisatos, A. P. Science 1998, 281, 2013-2016.

10. Willner, I.; Patolsky, F.; Wasserman, J. Angew. Chem. Int. Ed. 2001, 40, 1861-1864.

11. Kan, S. et al, Nature Mater., 2003, 2, 155.

### BACKGROUND OF THE INVENTION

Hybrid systems consisting of semiconductor quantum dots (QDs) coupled to biomaterials find growing interest in the developing research area of nanobiotechnology (1, 2). Photochemically-induced fluorescence resonance energy transfer (FRET) between molecular fluorophores (3-5) or the quenching of excited chromophores by metal nanoparticles (6) was reported to probe DNA hybridization processes, and, specifically, the formation and dissociation of hairpin structures. The replication of DNA on bulk surfaces was recently applied for the amplified bioelectronic detection of DNA (7), and the incorporation of redox-active units into the replicated DNA has enabled the electrochemical probing of the dynamics of replication (8).

The unique photophysical properties of semiconductor nanoparticles establish the possibility of applying semiconductor nanoparticles as efficient fluorescence labels (9) or as photoelectrochemical probes (10). Semiconductor nanocrystals have several advantages as FRET donors. The nanocrystals may be tailored, via control of size, composition and shape (11) to provide exceptional spectral coverage with symmetric emission profiles, enabling optimization of donor-acceptor spectral overlap. Additionally, due to their continuous absorption band they may be excited efficiently at shorter wavelength. Finally, the nanocrystals are significantly more stable emitters compared to the conventional dye molecules and as mentioned above, this is a critical feature for a feasible FRET microscopy scheme.

### SUMMARY OF THE INVENTION

The present invention provides an optical method and device for the determination of an analyte in an assayed sample. The method and device of the invention are based on the use of semiconductor nanoparticles that carry a recognition agent, thus forming a hybrid system. With this system, in the presence of an analyte and under assay conditions, a reaction occurs causing the immobilization of an acceptor to the recognition agent, either directly or through a reaction product of the recognition agent that is formed in the presence of the analyte.

The nanoparticles provide an active media with respect to electromagnetic radiation. The term “active media” is meant to denote a media capable of interacting with electromagnetic radiation resulting in absorption of the radiation followed by transfer of the energy to an acceptor.

Thus, the detection in the present invention is based on energy transfer between active media (donors) and acceptors. The acceptors accept energy from the donor, which stimulates electronic transitions to higher energy levels so that when they return to lower energy levels they emit energy, e.g. photonic radiation. More specifically, the detection is based on fluorescence resonance energy transfer (FRET) between semiconductor nanoparticle donors, which are excited with electromagnetic radiation, and acceptors in the form of dye-labeled agents, preferably dye-labeled nucleic acids or dye-labeled oligonucleotide sequences, nanoparticles-labeled agents such as nanoparticles-labeled nucleic acids or nanoparticles-labeled oligonucleotide sequences.

The term “donor” denotes a chemical entity having absorption and emission spectra. Typical donors in the present invention are semiconductor nanoparticles. The term “acceptor” denotes a chemical entity where a portion of its absorption spectrum is overlapping a portion of the emission spectrum of the donor such that the acceptor is capable of accepting energy from said donor. Typical acceptor molecules used in the present invention are dye molecules. Non limiting examples of dyes are Rhodamine based dyes, fluoresceines, cyanines, Texas red and other available dyes such as transition metal complexes acting as energy acceptors. Alternatively, the acceptor molecules are nanoparticles with acceptor spectral properties, e.g. InAs acceptors.

The donor nanoparticles are excited using a wavelength wherein absorption of the acceptor is negligible compared to that of the semiconductor nanoparticle. Efficient resonance energy transfer between the donor semiconductor nanoparticles and the acceptors would occur only when the acceptor agents are immobilized to the hybrid system comprising the nanoparticles and the recognition agent. Such attachment may happen only under appropriate reaction conditions and in the presence of the analyte. As used herein the terms “immobilize”, “attach ”and “bind”, at appropriate departures (e.g. attached, attaching, immobilized, immobilization, bound, binding, etc.) denote chemical binding (i.e. chemisorption, covalent linkage e.g. hydrogen bonds, Van der Waals bonds, etc.) or electrostatic linkage, e.g. ionic bonds, etc.

The term “reaction” is used to denote one or more reactions or interactions carried out at once or in sequence, to attach the acceptor, directly or indirectly, to the recognition agent. The “signal” in the context of the present invention is emission of light at a given wavelength corresponding to the emission wavelength of the acceptor. Accordingly, the term “assay conditions” encompasses all the conditions, substances or actions necessary or useful for the appropriate reaction to take place, including sequences of varying conditions or actions.

The recognition agent bound to the semiconductor nanoparticles may react with the analyte to form a complex. The complex may then be the attachment site for the acceptor, possibly by a catalyst. Alternatively, the analyte may be a catalyst that can induce a reaction in which the recognition agent is converted into a product. In this alternative, the signal would be present only if the catalyst converted the recognition agent.

The invention permits the qualitative detection of an analyte, namely to get a Yes/No answer whether the analyte exists in the assayed sample, as well as the quantitative detection, namely determine the presence as well as the level/amount of the analyte in the sample. In the following, the term “determination” or “determining” or “detection” will be used to refer collectively to both qualitative and quantitative assay of the analyte in the assayed sample.

Likewise, the present invention is not limited to the nature of the recognition agent and the analyte, the nature of the reaction or the assay conditions. Nevertheless, it is appreciated that the present invention is especially useful in the determination of DNA or RNA analytes, DNA polymerase or telomerase analytes, cancer cells through telomerase activity and single-base mismatches. In such cases the recognition agent is a single-stranded oligonucleotide.

Thus, according to one aspect of the invention, there is provided a method for determining an analyte in an assayed sample, comprising:
(a) providing semiconductor nanoparticles carrying a recognition agent capable of specifically binding to the analyte or undergoing a reaction in the presence of the analyte,
(b) contacting said semiconductor nanoparticles with the assayed sample;
(c) providing an acceptor capable of immobilization directly or indirectly, in the presence of the analyte, to the recognition agent;
(d) providing assay conditions, such that in the presence of the analyte in the assayed sample a reaction would occur, resulting in the direct or indirect immobilization of the acceptor to the recognition agent,
(e) irradiating the system so as to cause excitation of the semiconductor nanoparticles and energy transfer to the acceptor; and generation of an electromagnetic signal,
(f) detecting said signal,

whereby the signal is indicative of the presence and/or the amount of said analyte in the sample.

When the method is designed for quantitative detection, the detection comprises comparing the detected signal with a calibration curve showing the relation between the signal and known and varying amounts of analyte under said assay conditions, thereby determining the amount of said analyte in the sample.

The semiconductor nanoparticles are preferably nanocrystals having various shapes such as for example nanospheres, nanorods, quantum dots, branched structures such as tripods and tetrapods, tubes and wires.

Preferably, the semiconductor material is selected from Group II-VI semiconductors, such as for example CdS, CdSe, CdTe, ZnS, ZnSe, ZnO and alloys (e.g. CdZnSe); Group III-V semiconductors such as InAs, InP, GaAs, GaP, InN, GaN, InSb, GaSb and alloys (e.g., InAsP); Group IV-VI semiconductors such as PbSe and PbS and alloys; and Group IV semiconductors such as Si and Ge and alloys. Additionally, combinations of the above in composite structures consisting of sections with different semiconductor materials, for example CdSe/CdS or any other combinations, as well as core/shell structures of different semiconductors such as for example CdSe/ZnS core/shell nanoparticles or nanorods, are also within the scope of the present invention.

In a preferred embodiment the nanoparticles are in the form of quantum dots and even more preferably the nanoparticles are in the form of core-shell layered quantum dots (abbreviated “QD”). The signal generated is preferably emission of light.

The excitation of the semiconductor nanoparticles with electromagnetic radiation may be carried out at diverse wavelengths, preferably in the UV visible or IR range, depending on the sort of semi-conducting material used and on its form, e.g. nanoparticles, quantum dots, etc.

According to a preferred embodiment, the recognition agent and the analyte form a recognition couple and the detection of the analyte is based on the use of a reagent that binds to the formed couple. A specific example of such embodiment are cases where the analyte is a DNA analyte. In such cases, the assay conditions may comprise DNA polymerase and nucleotide bases, at least one of said nucleotide bases being bound to an acceptor. Experimental results show that the acceptor absorbs a certain wavelength of the energy emitted from the semiconductor nanoparticles upon irradiation of said nanoparticles with electromagnetic radiation, and emits light at a different wavelength.

The method of the invention may also provide the determination of at least one base mismatch. For example, the mutant sequence may have a single known base (the mutant base) replacing another base (being any one of the other three nucleotides) in a known position of the normal gene sequence. The recognition agent is a nucleic acid sequence that is shorter than the mutant sequence and the normal gene sequence, and is complementary to both of them up to one base prior to the mutation site, where said recognition agent terminates. Thus, hybridization of nanoparticle bound recognition agent with of any one of said DNAs leaves a single strand portion of said DNAs, beginning at said known position of the mutation.

The assay conditions in this example comprise a DNA polymerase and a dye-bound nucleotide that is complementary to the known base of the mutant sequence. Accordingly, the dye label would bind, through the DNA complex, to the nanoparticles only in complexes comprising the mutant sequence. Subsequently, irradiation of said nanoparticles with electromagnetic radiation would produce the dye specific signal only in case where the mutant analyte was present in the assayed sample This labeling may be intensified by application of thermal dissociation/annealing/labeling cycles, as part of the assay conditions.

A method for the detection of the presence of a specific base in a single stranded DNA analyte according to the present invention comprises the following steps:
(a) providing semiconductor nanoparticles carrying a DNA recognition agent that comprises a nucleic acid sequence that is complementary to a sequence in the DNA analyte up to one base prior to the specific base,
(b) contacting said semiconductor nanoparticles with the assayed sample,
(c) providing DNA polymerase and a nucleotide base, complementary to the specific base, bound to an acceptor,
(d) providing assay conditions that enable hybridization;
(e) irradiating the reaction mixture so as to cause excitation of the semiconductor nanoparticles, transfer of resonance energy from said nanoparticles to said acceptor and generation of a signal, and
(f) detecting said signal,
   whereby the signal is indicating the presence and/or amount of said specific base in the DNA analyte in the sample.

The specific base may be a single-base mutation. Alternatively the specific base may be a part of a longer sequence to be identified in the DNA analyte, and the presence of the base may serve as an indicator to the presence of the longer sequence.

The specific base may also serve to detect a specific SNP (single-base-polymorphism), that naturally occurs between individuals, for forensic reasons, for example for identifying suspects according to blood, semen, hair etc. left in a crime scene.

The method of the invention requires that the nanoparticles be excited in a region of wavelengths where absorption of the acceptor is negligible compared to that of the excited nanoparticles and under conditions where the emission of the nanoparticles overlap with the acceptor.

According to another embodiment, the analyte to be detected is a catalyst that can induce a reaction in which the recognition agent is converted into a product. A specific example of catalysts are enzymes, e.g. telomerase. The detection of telomerase activity is indicative of the presence of cancer cells. A method for detecting telomerase activity according to the present invention comprises:
(a) providing semiconductor nanoparticles carrying a single-stranded DNA recognition agent, that serves as a primer for telomerase reaction,
(b) providing an assay sample comprising cellular extract from one or more cells suspected of comprising telomerase,
(c) contacting said semiconductor nanoparticles with the assayed sample,
(d) providing nucleotide bases, at least one of said nucleotide bases being bound to an acceptor,
(e) providing assay conditions enabling telomerase-catalyzed DNA elongation reaction,
(f) irradiating the system so as to cause excitation of the semiconductor nanoparticles, transfer of resonance energy from said nanoparticles to said acceptor and generation of a signal, and
(g) detecting said signal,
   whereby the signal is indicating the presence and/or amount of cancer cells in the sample.

An alternative method for detecting telomerase activity according to the present invention comprises:
(a) providing semiconductor nanoparticles carrying a single-stranded DNA recognition agent, that serves as a primer for telomerase reaction,
(b) providing an assay sample comprising cellular extract from one or more cells suspected of comprising telomerase,
(c) contacting said semiconductor nanoparticles with the assayed sample and in the presence of nucleotide bases;
(d) providing assay conditions enabling telomerase-catalyzed DNA elongation reaction thereby producing telomere repeat units bound to said primer,
(e) providing a nucleotide sequence being complementary to the telomere repeat units and being bound to an acceptor,
(f) providing assay conditions giving rise to a hybridization reaction such that the nucleotide sequence of step (e) may bind to the telomere repeat units,
(g) irradiating the system so as to cause excitation of the semiconductor nanoparticles, transfer of resonance energy from said nanoparticles to said acceptor and generation of a signal, and
(h) detecting said signal,
   whereby the signal is indicating the presence and/or amount of said cells in the sample.

In a further alternative method for detecting telomerase activity, a single-stranded DNA recognition agent, that serves as a primer for telomerase reaction, is provided in solution. In such method the recognition agent is contacted with an assay sample comprising cellular extract from one or more cells suspected of comprising telomerase and nucleotide bases, thereby enabling telomerase-catalyzed DNA elongation reaction and binding of telomere repeat units to said recognition agent. In the next step the product of the elongation reaction is contacted with a nucleotide sequence being complementary to the telomere repeat units such that under assay conditions a hybridization reaction occurs. Each nucleotide sequence carries both donor and acceptor, where the distance between these two moieties is such that under irradiation conditions there is no energy transfer between the donors and the acceptors carried by the same sequence. In other words, energy transfer upon irradiation would occur only between the donor of one sequence and the acceptor of a neighboring sequence. Thus, irradiation of the system causes excitation of the donor nanoparticles of one sequence, transfer of resonance energy from said donor to the acceptor and generation of a signal. It has to be mentioned that transfer of resonance energy would not take place in the absence of said hybridization reaction. The detection of a signal is indicative of the presence and/or amount of telomerase in the sample.

According to another aspect, the present invention provides a sensing device for determining an analyte in an assayed sample, the device comprising an assay unit comprising a system of semiconductor nanoparticles carrying recognition agent and acceptor capable of immobilization, in the presence of the analyte and under assay conditions, to the recognition agent.

In a preferred embodiment, the above sensing device further comprises:
(i) irradiation unit for exciting said semiconductor nanoparticles, thereby generating a signal; and
(ii) measuring utility for detecting said signal.

In another aspect, the present invention provides a sensing device for determining the presence of two or more different analytes in an assayed sample, the system comprising (a1) a plurality of assay units, each unit for determining a specific analyte of the two or more different analytes, and having at least one unit for each of said different analytes, each of said units comprising (i) a system of semiconductor nanoparticles carrying recognition agents and (ii) acceptor capable of immobilization, in the presence of the analyte and under assay conditions, to the recognition agent. Each unit, e.g. spot produced by arrayer or spotter, may be found on different locations or regions of a single device.

In a preferred embodiment, the above device further comprises:
(i) irradiation unit for exciting said semiconductor nanoparticles, thereby generating a signal, and
(ii) measuring utility for detecting said signal.

The measuring utility in the above system is such that may identify which of the devices or regions in the system gave rise to the signal (and accordingly assist in the identification of the analyte(s) in the assayed sample) or measure the intensity of the signal, or both.

The above device, comprising an array of separate sensing devices is especially useful as a DNA array detection. The separate sensing devices are bound to a substrate by any surface modification technique. Thus, for example, if the substrate is made of gold or glass, the nanoparticles may be attached thereto through di-thiol linkers. The signals generated by the system may be read out by surface imaging techniques, where the emission of photons from areas bound to an analyte, is different from the emission from areas which did not bind an analyte.

In still another aspect, the present invention provides a kit for the detection of the presence or the amount of an analyte in an assayed sample comprising:
(a) semiconductor nanoparticles carrying a recognition agent;
(b) assay reagents comprising an acceptor capable to absorb the energy emitted by the semiconductor nanoparticles upon irradiation of said nanoparticles with electromagnetic radiation, thereby generating a signal;
(c) optionally a calibration curve showing the relation between the signal and the analyte amount under said assay conditions, thereby determining the amount of said analyte in the sample.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a scheme, which illustrates the telomerization and replication processes on CdSe—ZnS quantum dots functionalized with nucleic acids and with the incorporation of Texas Red-labeled dUTP.

FIG. 2A shows the emission spectra upon the time-dependent telomerization on the CdSe—ZnS QDs: (i)—before addition of telomerase, (ii), (iii) and (iv): after 10, 30 and 60 minutes of telomerization, respectively.

FIG. 2B shows the AFM image of CdSe—ZnS QDs before telomerase treatment.

FIG. 2C shows the AFM image of a CdSe—ZnS QD after 60 minutes telomerization (images in FIGS. 2A and 2B are recorded on mica surfaces activated with 5 mM MgCl2).

FIG. 3A shows the emission spectra upon the time-dependent DNA-Replication on the CdSe—ZnS QDs: (i)—before addition of dye-dUTP, (ii), (iii) and (iv): after 1, 30 and 60 minutes of replication, respectively.

FIG. 3B shows the AFM image of CdSe—ZnS quantum dots hybridized with M13φ DNA.

FIG. 4 is a scheme, which illustrates another telomerization processes on semiconductor quantum dots functionalized with nucleic acids.

FIG. 5 is a scheme which illustrates a further telomerization process where the semiconductor quantum dots and acceptor dye are immobilized on an oligonucleotide sequence.

### DETAILED DESCRIPTION OF THE INVENTION

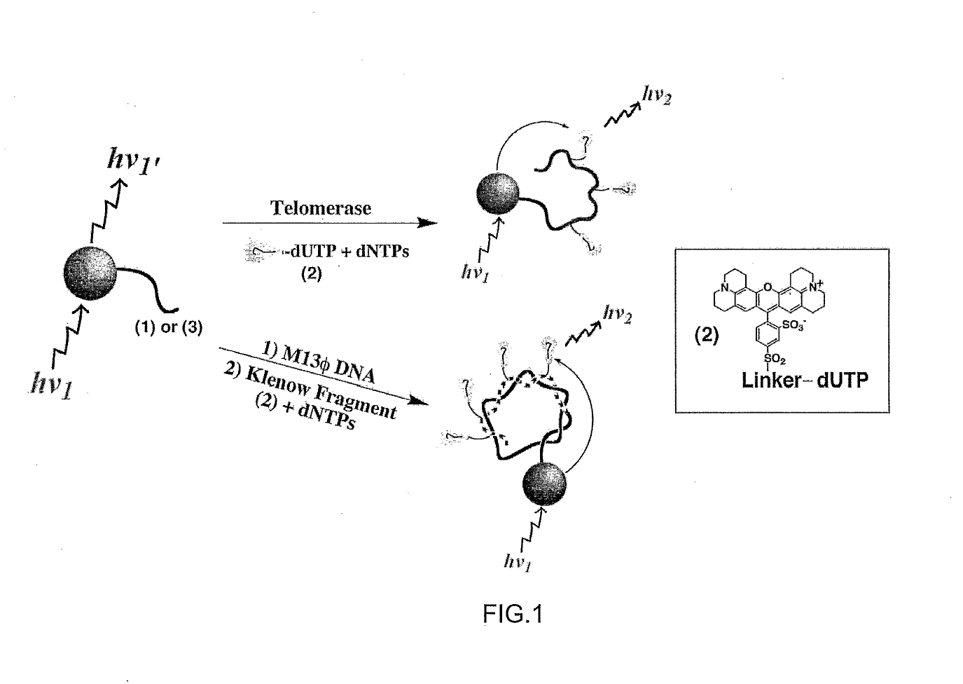

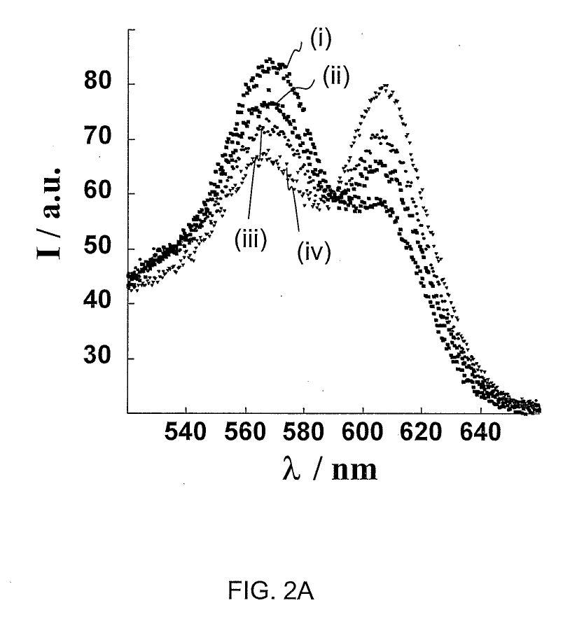

A preferred embodiment of the method of the present invention is schematically illustrated in FIG. 1. CdSe—ZnS biocompatible quantum dots (hereinafter abbreviated “QDs”) were used as photochemical centers for telomerization or DNA replication occurring on the particles of a telomerase-recognized nucleic acid or a DNA (or RNA) hybridized to a nucleic acid associated to a quantum dot in the presence of a telomerase or a polymerase or reverse transcriptase in the presence of all nucleotide mixture that includes a dye-labeled nucleotide such as dye-labeled dUTP or dCTP. The dye units incorporated into the new synthesized telomer or DNA replica enable fluorescence energy transfer (FRET) from the excited particles to the dye and the observation of the fluorescence characteristic to the dye. For the telomerase analysis, according to FIG. 1, CdSe—ZnS nanoparticles (4.2 nm diameter, λₑₘ=400 nm, 20% luminescence quantum yield) stabilized by the mercaptopropionic ligand were modified with the thiolated oligonucleotide (1) (sequence: 5′-HS-(CH₂)₆-TTTTTTAATCCGTCGAGCAGAGTT-3′). Analysis of the nanoparticles indicated that ca. 25 nucleic acid units were associated with each nanoparticle. The (1)-functionalized semiconductor QDs were incubated with a dNTP mixture (dATP, nucleotide Texas-Red 14-dUTP, (2)), (100 μM) in the presence of telomerase (extracted from HeLa cells, 10.000 cells). FIG. 2A shows the fluorescence spectra of the system upon excitation of the CdSe—ZnS QDs at λ=400 nm, as a function of telomerization time. The CdSe—ZnS QDs prior to the introduction of telomerase emitted at λ=560 nm. After the addition of telomerase, and as telomerization proceeds, the fluorescence of the QDs decreased, with the concomitant increase of the characteristic emission of the dye at λ=610 nm.

Control experiments revealed that excitation of the nucleotide mixture that includes the dye modified dUTP in the absence of the CdSe—ZnS QDs at λ=400 nm does not lead to an observable emission at λ=560 nm. However, excitation of the system at λ=550 nm lead to a high fluorescence signal characteristic to the dye. As the emission of the CdSe—ZnS QDs at λmax=560 nm coincides with the absorbency band of the dye (2), the emission observed upon telomerization is attributed to fluorescence resonance energy transfer (FRET) from the nanoparticles to the dye molecules incorporated into the telomeric units by telomerase.

Atomic Force Microscopy (AFM) images of the CdSe—ZnS QDs prior to the telomerization, FIG. 2B, and the image of a nanoparticle after telomerization, FIG. 2C were recorded on freshly cleaved mica surfaces, under ambient conditions. While the height of the nanoparticle is ca. 4 nm, its lateral dimensions are distorted due to the tip dimensions. The nanoparticle after telomerase incubation reveals beautifully the synthesized hinged DNA strands. The height of the telomeric DNA chains is ca. 1 nm, whereas their length is ca. 300 nm. This corresponds to a telomerase induced elongation of ca. 1000 base units in the telomeric DNA chain.

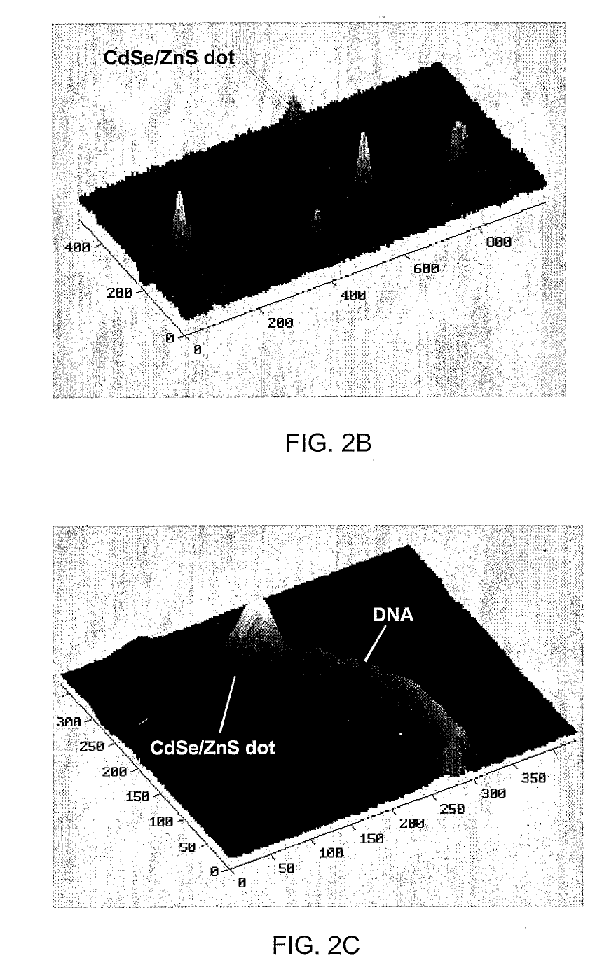

The CdSe—ZnS quantum dots enabled also the probing of the dynamics of polymerase replication of DNA, as showed schematically in FIG. 1. In this system the primer (3) (sequence: ‘5-HS-(CH₂)₆-CCCCCACGTTGTAAAACGACGGCCAGT-3’) complementary to M13φDNA was assembled on the CdSe—ZnS QDs. Hybridization of the (3)-functionalized QDs with M13φDNA followed by replication in the presence of polymerase (Klenow fragment, 10 units) and a mixture of dNTPs containing the fluorophore-labeled nucleotide, yield the dye-labeled DNA replica. The dynamics of DNA replication could be followed by FRET from the luminescent QDs to the incorporated dye unit, as shown in FIG. 3A. An AFM image of two CdSe—ZnS nanoparticles hybridized to M13φDNA is shown in FIG. 3B.

The method and device of the invention may be applied for the fast and sensitive detection of cancer cells and as an amplification route for analyzing DNA on chip arrays. The polymerase-induced replication of DNA on the surface of luminescent QDs represents an “AND” gate where FRET readout occurs only if hybridization and replication proceed.

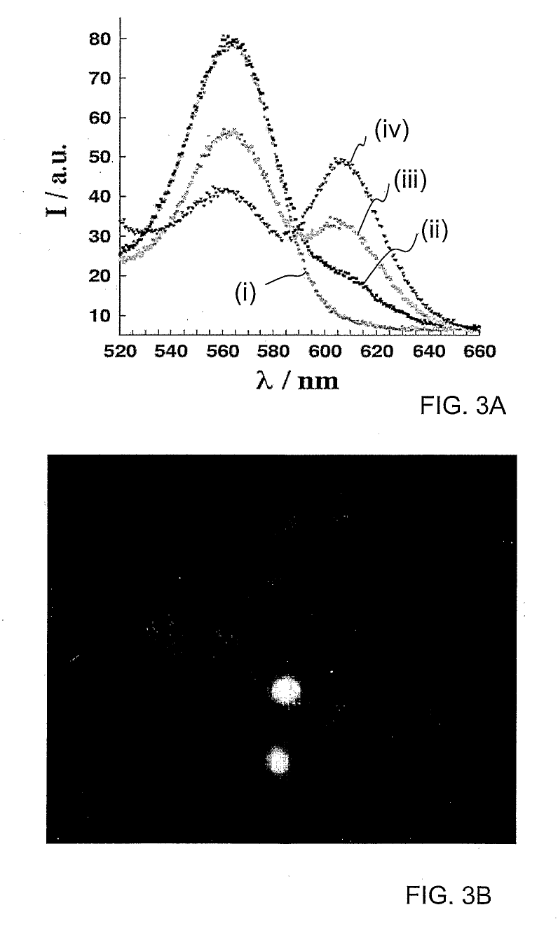

Another preferred embodiment of the invention is schematically showed in FIG. 4. According to this embodiment, semiconductor nanoparticles (SC) carrying a single-stranded DNA recognition agent, that serves as a primer for telomerase reaction are contacted, in the presence of nucleotide bases, with an assay sample suspected of comprising telomerase. Under proper assay conditions, telomerase-catalyzed. DNA elongation reaction occurs, thus producing telomere repeat units bound to the primer. In a subsequent step there is provided a nucleotide sequence being complementary to the telomere repeat units and being bound to an acceptor (A). A hybridization reaction takes place thus resulting in the immobilization of the acceptor to the complementary part of the telomere repeat units. Irradiation of the system causes excitation of the semiconductor nanoparticles (SC), transfer of resonance energy from the nanoparticles to the acceptor (A) and generation of an optical signal, where the signal is indicating the presence and/or amount of telomerase in the sample.

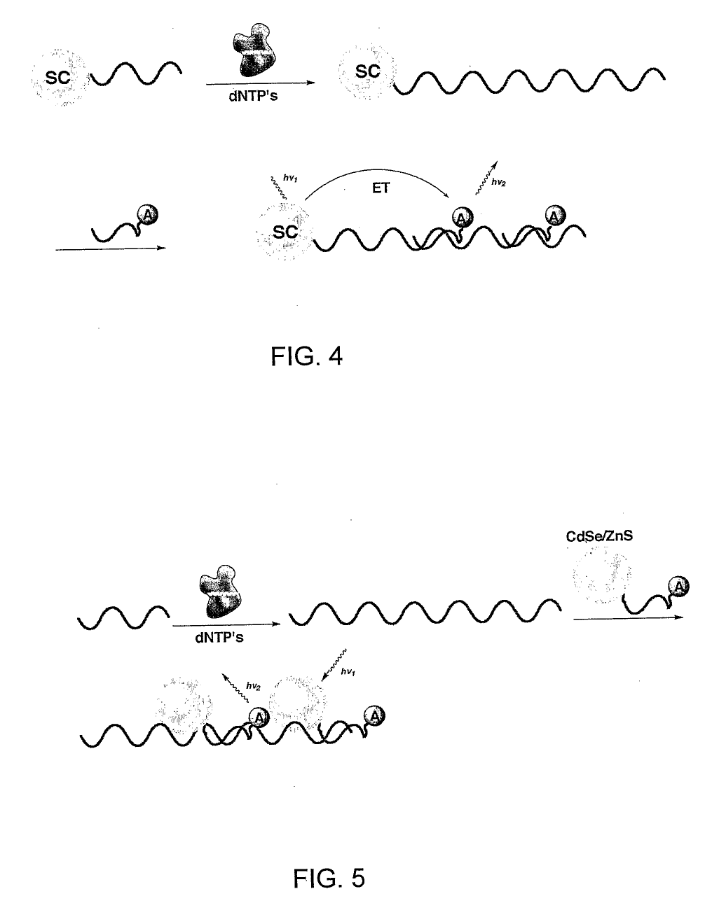

In a further alternative embodiment, schematically showed in FIG. 5, for detecting telomerase activity, a single-stranded DNA recognition agent, that serves as a primer for telomerase reaction, is provided in solution. In this method the recognition agent is contacted with an assay sample suspected of comprising telomerase. In the presence of nucleotide bases a telomerase-catalyzed DNA elongation reaction occurs, resulting in the binding of telomere repeat units to the recognition agent. In the next step the product of the elongation reaction is contacted with a nucleotide sequence being complementary to the telomere repeat units such that under assay conditions a hybridization reaction occurs. Each nucleotide sequence carries both donor and acceptor, where the distance between these two moieties is such that under irradiation conditions there is no energy transfer between the donors and the acceptors carried by the same sequence. In other words, energy transfer upon irradiation would occur only between the donor of one sequence and the acceptor of a neighboring sequence. Thus, irradiation of the system causes excitation of the donor nanoparticles of one sequence, transfer of resonance energy from said donor to the acceptor and generation of a signal. It has to be mentioned that transfer of resonance energy would not take place in the absence of said hybridization reaction. The detection of a signal is indicative of the presence and/or amount of telomerase in the sample.

### EXAMPLES

### Synthesis of Functionalized Nanoparticles

Synthesis of CdSe—ZnS nanoparticles: trioctylphospine oxide (TOPO) protected nanoparticles, soluble in organic solvents were reacted in toluene (1 ml) with 100 μl of mercaptopropionic acid overnight. (Protected from light with aluminum foil). After this incubation time the nanoparticles were transferred to water by adding 1M KOH solution (1 ml), the two phase mixture was separated and this process repeated a second time with clean toluene (1 ml). The water-soluble CdSe—ZnS nano-particles were separated from the excess of mercaptopropionic acid by 3 repeated cycles of precipitation of the nanoparticles with acetone and followed by the re-dissolution of the nanoparticles in a phosphate buffer solution pH 7.4, 10 mM.

Preparation of DNA-modified CdSe—ZnS nanoparticles: The mercaptopropionic acid functionlized CdSe—ZnS nanoparticles (0.1 O.D) were reacted with freshly reduced and purified thiolated oligonucleotides ((1) or (3)) 6-10 O.D) overnight in order to allow the exchange of the thiol group associated with the nanoparticles with thiolated oligonucleotide. The DNA modified nanoparticles were separated from the free oligonucleotides by the use of micro-spin filter (Millipore, 30 KDa) or by acetone precipitation and re-dissolution in phosphate buffer solution. The nucleic acid-modified nanoparticles were obtained with a 70% yield. The fluorescence quantum yield of the nucleic acid-functionalized nanoparticles is ca. 15%.

1. A method for determining an analyte in an assayed sample, comprising:
(a) providing semiconductor nanoparticles carrying a recognition agent capable of specifically binding to the analyte or undergoing a reaction in the presence of the analyte,
(b) contacting said semiconductor nanoparticles with the assayed sample;
(c) providing an acceptor capable of immobilization directly or indirectly, in the presence of the analyte, to the recognition agent;
(d) providing assay conditions, such that in the presence of the analyte in the assayed sample a reaction would occur, resulting in the direct or indirect immobilization of the acceptor to the recognition agent,
(e) irradiating the system so as to cause excitation of the semiconductor nanoparticles and energy transfer to the acceptor; and generation of an electromagnetic signal,
(f) detecting said signal,
whereby the signal is indicative of the presence and/or the amount of said analyte in the sample.

2. The method of claim 1 wherein said nanoparticles are in the form of quantum dots.

3. The method of claim 1, wherein said signal is emission of light.

4. The method of claim 1 wherein the recognition agent and the analyte form a recognition couple and the detection of the analyte is based on the use of a reagent that binds to the formed couple.

5. The method of claim 4, wherein said analyte is a DNA analyte.

6. The method of claim 5, wherein the assay conditions comprise DNA polymerase and nucleotide bases, at least one of said nucleotide bases being bound to an acceptor.

7. The method according to claim 1, wherein said acceptor is selected from dye-labeled nucleic acids, dye-labeled oligonucleotide sequences, nanoparticles-labeled nucleic acids and nanoparticles-labeled oligonucleotide sequences.

8. The method of claim 1, wherein said nanoparticles are excited in a region where absorption of the acceptor is negligible compared to that of the nanoparticles.

9. The method of claim 1, wherein said acceptor is a fluorescent dye.

10. The method of claim 1, wherein the acceptor is semiconductor nanoparticle.

11. The method of claim 5, wherein the analyte is a nucleotide sequence having at least one base mutation.

12. The method according to claim 11, wherein the assay conditions comprise DNA polymerase and a nucleotide base complementary to the single base mutation and being bound to an acceptor selected from dye moiety and semiconductor nanoparticle.

13. The method of claim 1 wherein the analyte is a catalyst that can induce a reaction in which the recognition agent is converted into a product.

14. (canceled)

15. The method of claim 13, wherein the enzyme is telomerase.

16. (canceled)

17. The method of claim 15 for the detection of cancer cells.

18. The method of claim 15 comprising:
(a) providing semiconductor nanoparticles carrying a single-stranded DNA recognition agent, that serves as a primer for telomerase reaction,
(b) providing an assay sample comprising cellular extract from one or more cells suspected of comprising telomerase;
(c) contacting said semiconductor nanoparticles with the assayed sample;
(d) providing nucleotide bases, at least one of said nucleotide bases being bound to an acceptor
(e) providing assay conditions that give rise to a DNA elongation reaction,
(f) irradiating the system so as to cause excitation of the semiconductor nanoparticles, transfer of resonance energy from said nanoparticles to said acceptor and generation of a signal, and
(g) detecting said signal,
whereby the signal is indicating the presence and/or amount of telomerase in the sample.

19. A method according to claim 15 comprising:
(a) providing semiconductor nanoparticles carrying a single-stranded DNA recognition agent, that serves as a primer for telomerase reaction,
(b) providing an assay sample comprising cellular extract from one or more cells suspected of comprising telomerase,
(c) contacting said semiconductor nanoparticles with the assayed sample and in the presence of nucleotide bases;
(d) providing assay conditions enabling telomerase-catalyzed DNA elongation reaction thereby producing telomere repeat units bound to said primer,
(e) providing a nucleotide sequence being complementary to the telomere repeat units and being bound to an acceptor,
(f) providing assay conditions giving rise to a hybridization reaction such that the nucleotide sequence of step (e) may bind to the telomere repeat units,
(g) irradiating the system so as to cause excitation of the semiconductor nanoparticles, transfer of resonance energy from said nanoparticles to said acceptor and generation of a signal, and
(h) detecting said signal,
whereby the signal is indicating the presence and/or amount of telomerase in the sample.

20. A method according to claim 15 comprising:
(a) providing a solution comprising a single-stranded DNA recognition agent, that serves as a primer for telomerase reaction,
(b) contacting said solution with nucleotide bases and an assay sample comprising cellular extract from one or more cells suspected of comprising telomerase and nucleotide bases, thereby enabling telomerase-catalyzed DNA elongation reaction and binding of telomere repeat units to said recognition agent,
(c) contacting the product of step (b) with a nucleotide sequence carrying both semiconductor nanoparticles donor and acceptor, said sequence being complementary to the telomere repeat units such that under assay conditions a hybridization reaction occurs,
(d) irradiating the system so as to cause excitation of the donor of one sequence and transfer of resonance energy from said donor to the acceptor of a neighboring sequence and generation of a signal, and
(e) detecting said signal,
whereby the signal is indicating the presence and/or amount of telomerase in the sample.

21. A sensing device for determining a specific analyte in an assayed sample, the device comprising assay unit comprising a system of semiconductor nanoparticles carrying recognition agent and acceptor capable of immobilization, in the presence of the analyte and under assay conditions, to the recognition agent.

22. (canceled)

23. (canceled)

24. The device of claim 21 wherein excitation of the semiconductor nanoparticles is by electromagnetic radiation.

25. (canceled)

26. (canceled)

27. (canceled)

28. (canceled)

29. (canceled)

30. A sensing device for determining the presence of two or more different analytes in an assayed sample, the system comprising a plurality of assay units, each unit for determining a specific analyte of the two or more different analytes, and having at least one unit for each of said different analytes, each of said units comprising a system of semiconductor nanoparticles carrying recognition agents and acceptor capable of immobilization, in the presence of the analyte and under assay conditions, to the recognition agent.

31. (canceled)

32. A kit for the detection of the presence or the amount of an analyte in an assayed sample comprising:
(a) semiconductor nanoparticles carrying a recognition agent;
(b) assay reagents comprising an acceptor capable to absorb the energy emitted by the semiconductor nanoparticles upon irradiation of said nanoparticles with electromagnetic radiation, thereby generating a signal
(c) optionally a calibration curve showing the relation between the signal and the analyte amount under said assay conditions, thereby determining the amount of said analyte in the sample.

33. The kit according to claim 32 wherein said recognition agent is a single-stranded oligonucleotide.

34. The kit according to claim 32 wherein said analyte is a DNA analyte and said assay conditions comprise DNA polymerase and nucleotide bases, at least one of said nucleotide bases being bound to an acceptor.

35. The kit according to claim 32 wherein said analyte is a nucleotide sequence having at least one base mutation and said assay conditions comprise DNA polymerase and a nucleotide base complementary to the single base mutation and being bound to an acceptor.

36. The kit according to claim 32 wherein said analyte is telomerase, said recognition agent is a single-stranded DNA that serves as a primer for telomerase reaction and said assay conditions comprise nucleotide bases, where at least one of said nucleotide bases is bound to an acceptor molecule.
